(19) United States
(12) Patent Application Publication
(10) Pub. No.: US 2025/0143546 A1
(43) Pub. Date: May 8, 2025

(54) MEDICAL DEVICES WITH MODULAR COMPONENTS AND ASSEMBLIES

(71) Applicant: Boston Scientific Scimed, Inc., Maple Grove, MN (US)

(72) Inventors: George Wilfred DUVAL, Sudbury, MA (US); Louis J. BARBATO, Franklin, MA (US); James WELDON, Newton, MA (US)

(73) Assignee: Boston Scientific Scimed, Inc., Maple Grove, MN (US)

(21) Appl. No.: 18/929,033

(22) Filed: Oct. 28, 2024

Related U.S. Application Data
(60) Provisional application No. 63/595,463, filed on Nov. 2, 2023.

(51) Int. Cl. A61B 1/00; A61B 1/05; A61B 1/06

(52) U.S. Cl. CPC A61B 1/00124; A61B 1/00101; A61B 1/00105; A61B 1/053; A61B 1/0676

(57) ABSTRACT

A medical device includes a shaft, a printed circuit board positioned around an outer surface of the shaft, and an end cap. The printed circuit board includes a plurality of electrical connections at a proximal end of the printed circuit board and another plurality of electrical connections at a distal end of the printed circuit board. The printed circuit board includes a plurality of communication conduits connecting electrical connections at the proximal end of the printed circuit board with respective electrical connections at the distal end of the printed circuit board. When the end cap is coupled to the shaft, (1) one or more cap lumens are in communication with one or more lumens in the shaft, and (2) the plurality of end cap electrical connections are electrically connected to the electrical connections at the distal end of the printed circuit board.

### CROSS-REFERENCE TO RELATED APPLICATIONS

This application claims the benefit of priority under 35 U.S.C. § 119 from U.S. Provisional Application No. 63/595,463, filed Nov. 2, 2023, which is incorporated by reference herein in its entirety.

### TECHNICAL FIELD

Various aspects of this disclosure relate generally to medical device with modular components and assemblies. In particular, aspects of this disclosure pertain to an outer flex printed circuit board wrap and a distal end cap for use with medical devices, among other aspects.

### BACKGROUND

A medical device (e.g., a duodenoscope, an endoscope, a bronchoscope, an endoscopic ultrasonography (“EUS”) scope, etc.) may be used for an endoscopy procedure, where the medical device comes in contact with a patient. The manufacturing process for the medical device often includes pulling or otherwise positioning a plurality of cables and/or wires through a lumen or working channel of a shaft of the medical device. This often results in a complicated and/or time-consuming manufacturing process. Moreover, positioning the cables and/or wires in a lumen or working channel of the shaft may also reduce the amount of space in the lumen or working channel of shaft.

The devices, components, and assemblies of this disclosure may help to rectify one or more of the deficiencies described above and/or address other aspects of the art.

### SUMMARY

Each of the aspects disclosed herein may include one or more of the features described in connection with any of the other disclosed aspects.

In an example, a medical device may comprise a shaft, wherein the shaft may include one or more lumens extending longitudinally from a proximal end of the shaft to a distal end of the shaft; a printed circuit board, wherein the printed circuit board may be flexible and may be positioned around an outer surface of the shaft, wherein the printed circuit board may include a plurality of electrical connections at a proximal end of the printed circuit board, wherein the printed circuit board may include another plurality of electrical connections at a distal end of the printed circuit board, wherein the printed circuit board may include a plurality of communication conduits connecting electrical connections at the proximal end of the printed circuit board with respective electrical connections at the distal end of the printed circuit board; and an end cap, wherein the end cap may include one or more cap lumens and a plurality of end cap electrical connections, wherein, when the end cap is coupled to the shaft, (1) the one or more cap lumens may be in communication with the one or more lumens in the shaft, and (2) the plurality of end cap electrical connections may be electrically connected to the electrical connections at the distal end of the printed circuit board.

Any of the devices disclosed herein may include any of the following features, additionally or alternatively, in any combination. The medical device may include one or more integrated circuits coupled to and in electrical communication with the printed circuit board. A portion of the shaft may include a groove, and the one or more integrated circuits may be positioned within the groove. The groove may have a width and a length that are greater than a width and a length of the one or more integrated circuits. The groove may be positioned within a distal portion of the shaft. The printed circuit board may be configured to connect to the shaft via an operator applying pressure on the printed circuit board, so that the one or more integrated circuits attached to the printed circuit board and the shaft snap together. The medical device may include one or more capacitors or resistors coupled to and in electrical communication with the printed circuit board. The medical device may include an anisotropic conductive film, wherein the anisotropic conductive film secures the one or more integrated circuits to the printed circuit board. The one or more lumens in the shaft may include a working channel, an irrigation lumen, a suction lumen, and a plurality of pull wire lumens. The medical device may include a handle coupled to the proximal end of the shaft, wherein the handle includes a port fluidly connected to the working channel of the shaft.

The end cap may include one or more lighting sources and an imaging device, wherein the one or more lighting sources and the imaging device receive power via the plurality of electrical connections at the distal end of the shaft. The end cap may include an end cap printed circuit board that includes the plurality of end cap electrical connections. The plurality of communication conduits may include one or more of: one or more cables, one or more wires, one or more optical fibers, or one or more illumination fibers for transmitting electrical signals. The end cap may be configured to removably attach to the distal end of the shaft via a snap fit. A portion of the printed circuit board may be positioned around the outer surface with a larger longitudinal spacing between at least two portions of the printed circuit board, and a different section of the printed circuit board may be positioned around the outer surface with a smaller longitudinal spacing between at least two other portions of the printed circuit board.

In another example, a medical device may comprise a shaft, wherein the shaft may include one or more lumens extending longitudinally from a proximal end of the shaft to a distal end of the shaft, wherein a portion of the shaft may include a groove, and wherein one or more integrated circuits may be positioned within the groove; and a flexible printed circuit board positioned around an outer surface of the shaft, the flexible printed circuit board may include a plurality of proximal electrical connections on the proximal end of the shaft and a plurality of distal electrical connections on the distal end of the shaft, wherein a plurality of electrical communication conduits may connect the plurality of proximal electrical connections and the plurality of distal electrical connections.

Any of the devices disclosed herein may include any of the following features, additionally or alternatively, in any combination. The shaft may include a lubricious layer around the outer surface of the shaft. The groove may extend radially inward relative to the outer surface of the shaft, and wherein the groove may have a width and a length that are greater than an integrated circuit width and an integrated circuit length of the one or more integrated circuits.

In another example, a medical device may comprise a shaft, wherein the shaft may include one or more lumens extending longitudinally from a proximal end of the shaft to a distal end of the shaft, and wherein the shaft may include one or more electrical connections attached to an outer surface of the shaft; and an end cap removably coupled to the distal end of the shaft, wherein the end cap may include one or more cap lumens and a plurality of end cap electrical connections, wherein, when the end cap is coupled to the shaft, (1) the one or more cap lumens may be in communication with the one or more lumens in the shaft, and (2) the plurality of end cap electrical connections may be electrically connected to the one or more electrical connections of the shaft.

Any of the devices disclosed herein may include any of the following features, additionally or alternatively, in any combination. The end cap may include a flexible printed circuit board, wherein, when the end cap is coupled to the shaft, one or more electrical connections of the flexible printed circuit board may be electrically connected to the one or more electrical connections attached to the outer surface of the shaft.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. Several drawings include arrows labeled “P” and “D,” indicating proximal and distal directions, respectively. The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A-B depict an exemplary medical device, according to this disclosure.

FIG. 2A depicts a side view of an exemplary shaft of a medical device, according to this disclosure.

FIG. 2B depicts a distal end view of an exemplary shaft of a medical device of FIG. 2A, according to this disclosure.

FIG. 3 depicts another exemplary shaft of a medical device, according to this disclosure.

FIGS. 4A-C depict various aspects of an exemplary distal cap and an exemplary shaft of a medical device, according to this disclosure.

FIG. 5 depicts another exemplary distal cap for use with another exemplary shaft of a medical device, according to this disclosure.

### DETAILED DESCRIPTION

As discussed in the Background, a medical device (e.g., a duodenoscope, an endoscope, a bronchoscope, an endoscopic ultrasonography (“EUS”) scope, etc.) may be used for an endoscopy procedure, where the medical device comes in contact with a patient. The manufacturing process for the medical device often includes pulling or otherwise positioning a plurality of cables and/or wires through a lumen or working channel of a shaft of the medical device. This often results in a complicated and/or time-consuming manufacturing process. Moreover, positioning the cables and/or wires in a lumen or working channel of the shaft may also reduce the amount of space in the lumen or working channel of shaft. As a result, a need exists for devices, components, and assemblies for reducing the cables and wires that are pulled through the lumen of the shaft during the manufacturing process.

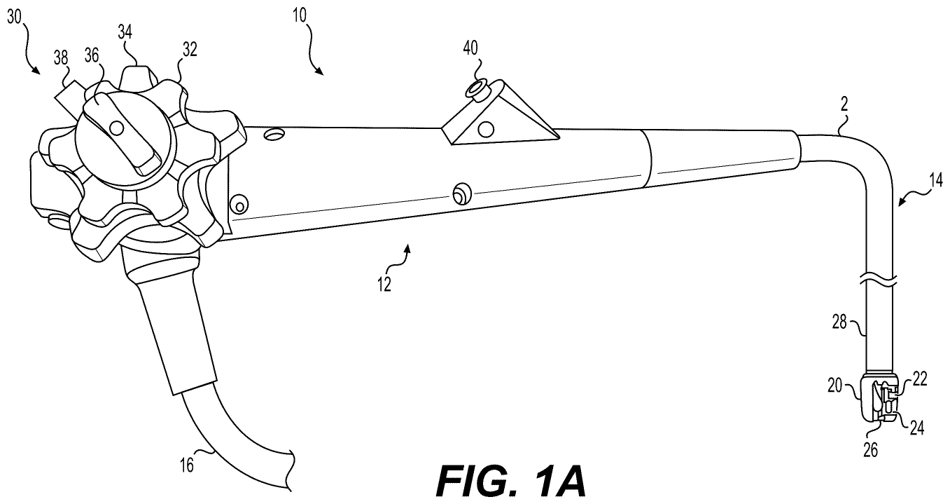

FIG. 1A depicts an exemplary duodenoscope 10 having a handle 12 and an insertion portion 14. FIG. 1B shows a proximal end of the handle 12. The duodenoscope 10 may also include an umbilicus 16 for purposes of connecting the duodenoscope 10 to sources of, for example, air, water, suction, power, etc., as well as to image processing and/or viewing equipment. Although a duodenoscope may be referenced herein, it will be appreciated that the disclosure also encompasses endoscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

The insertion portion 14 may include a sheath or shaft 2 and a distal tip 20. The distal tip 20 may include an imaging device 22 (e.g., a camera) and a lighting source 24 (e.g., an LED or an optical fiber). The distal tip 20 may be side-facing. That is, the imaging device 22 and the lighting source 24 may face radially outward, perpendicularly, approximately perpendicularly, or otherwise transverse to a longitudinal axis of the shaft 2 and the distal tip 20. Additionally or alternatively, the distal tip 20 may include one or more imaging devices 22 that face in more than one direction. For example, a first imaging device 22 may face radially outward, and a second imaging device (not shown) may face distally (approximately parallel to a longitudinal axis of the distal tip 20/shaft 2). In some embodiments, each imaging device (e.g., first imaging device 22) may include a lighting source (e.g., lighting source 24).

The distal tip 20 may also include an elevator 26 for changing an orientation of a tool inserted in a working channel of the duodenoscope 10. The elevator 26 may alternatively be referred to as a swing stand, pivot stand, raising base, or any suitable other term. The elevator 26 may be pivotable via, for example, an actuation wire or another control element that extends from the handle 12, through the shaft 2, to the elevator 26.

A distal portion of shaft 2 that is connected to the distal tip 20 may have a steerable section 28. The steerable section 28 may be or include, for example, an articulation joint. The shaft 2 and the steerable section 28 may include a variety of structures that are known or may become known in the art.

The handle 12 may have one or more actuators/control mechanisms 30. The control mechanisms 30 may provide control over the steerable section 28, the elevator 26, and/or may allow for provision (e.g., delivery or application) of air, water, suction, etc. For example, the handle 12 may include the control knobs 32, 34 for left, right, up, and/or down control of a steerable section 28. For example, one of the knobs 32, 34 may provide left/right control of the steerable section 28, and the other of the knobs 32, 34 may provide up/down control of the steerable section 28. The handle 12 may further include one or more of the locking mechanisms 36 (e.g., knobs or levers) for preventing steering and/or braking of the steerable section 28 in at least one of an up, down, left, or right direction. The handle 12 may include an elevator control lever 38 (see FIG. 1B). The elevator control lever 38 may raise and/or lower the elevator 26, via connection between the lever 38 and an actuating wire that extends from the elevator control lever 38, through the shaft 2, to the elevator 26. A port 40 may allow passage of a tool through the port 40, into a working channel of the duodenoscope 10, through the sheath 2, to the distal tip 20.

In use, an operator may insert at least a portion of the shaft 2 into a body lumen of a subject. The distal tip 20 may be navigated to a procedure site in the body lumen. The operator may insert an accessory device (not shown) into the port 40, and may pass the accessory device through the shaft 2 via a working channel to the distal tip 20. The accessory device may exit the working channel at the distal tip 20. The user may use the elevator control lever 38 to raise the elevator 26 and angle the accessory device toward a desired location (e.g., a papilla of the pancreatic-biliary tract). The user may use the accessory device to perform a medical procedure.

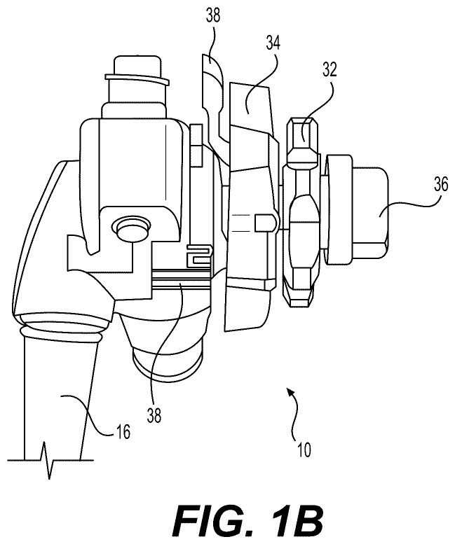

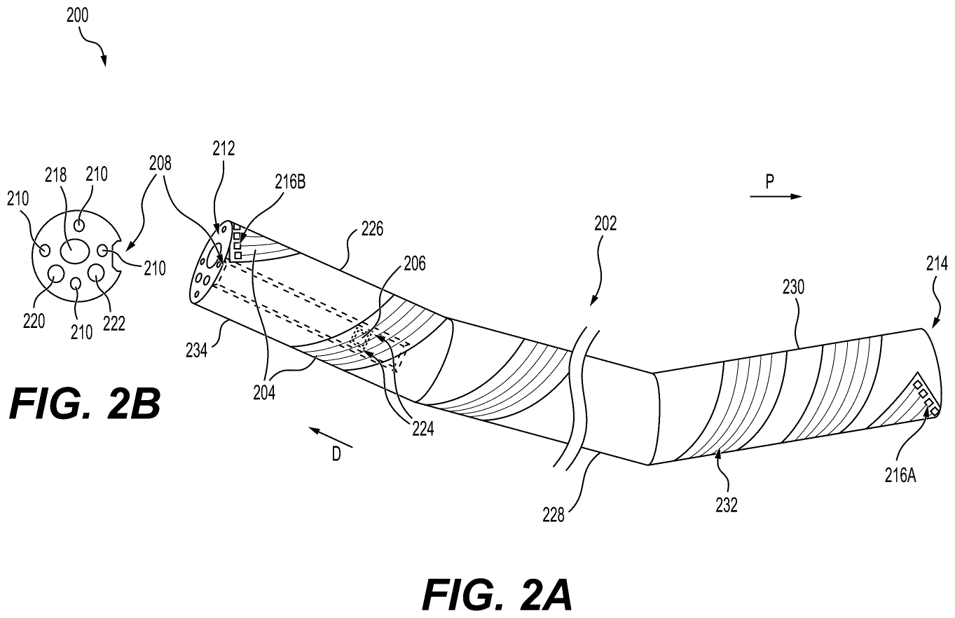

FIG. 2A depicts an embodiment of a side view of a shaft 202 that could be used for shaft 2 in the embodiment shown in FIG. 1A. FIG. 2B depicts an embodiment of a distal end view of shaft 202. The shaft 202 may comprise the same (or similar) elements as the shaft 2 of FIG. 1A.

The shaft 202 includes a proximal end 214 and a distal end 212. The proximal end 214 connects to the handle 12 (FIG. 1A) using any suitable connection, such as via welding, a friction fit, a snap fit, and the like. The distal end 212 connects to a distal tip (e.g., the distal tip 20 shown in FIG. 1A) using any suitable connection, such as via welding, a friction fit, a snap fit, and the like. In some embodiments, as discussed below, the distal tip may include a distal cap, and the distal end 212 may be connected to the distal cap using any suitable connection.

The shaft 202 includes one or more lumens that extend from the proximal end 214 to the distal end 212. One or more of the lumens may allow for sources of, for example, air, water, suction, power, etc., to pass from the handle 12 (FIG. 1A), through the shaft 202, to the distal tip 20. The one or more lumens may include one or more steering wire lumens 210, a working channel 218, a suction lumen 220, and an irrigation lumen 222. Although FIG. 2B illustrates shaft 202 including each of steering wire lumens 210, a working channel 218, a suction lumen 220, and an irrigation lumen 222, it is noted that shaft 202 may include only a subset (e.g., one, two, three, etc.) of these lumens or channels. An operator may insert an accessory device (not shown) into a port (e.g., the port 40) of the handle 12, and pass the accessory device through the shaft 202 via the working channel 218 to the distal end 212. The accessory device may exit the working channel 218 at the distal end 212. The operator may use the accessory device to perform a medical procedure. Upon conclusion of the procedure, the accessory device may be removed from the working channel 218. The suction lumen 220 is a lumen that extends through the shaft 202 and provides suctioning or negative pressure functionality at the distal end 212 of the shaft 202, for example, to help remove fluid, material, tissue, etc. from a treatment site. The irrigation lumen 222 is a lumen that extends through the shaft 202 and provides irrigation functionality at the distal end 212 of the shaft 202, such as for cleaning an imaging device, clearing the treatment site for better visualization, etc.

The shaft 202 includes a distal shaft portion 226, an intermediate shaft portion 228, and a proximal shaft portion 230. The distal shaft portion 226 is a portion of the shaft 202 located the furthest away from the handle 12. The proximal shaft portion 230 is a portion of the shaft 202 that connects to the handle 12. The intermediate shaft portion 228 is a portion of the shaft 202 that connects the distal shaft portion 226 and the proximal shaft portion 230.

The shaft 202 may be generally cylindrical. Additionally, a portion of the shaft 202 may include one or more coatings or layer, for example, a lubricious layer. In some aspects, shaft 202 may include one or more jackets (e.g., a jacket braid) on or around an exterior or outer surface 234 of shaft 202. As shown in FIG. 2A, shaft 202 includes a printed circuit board (PCB) wrap 204, which may be at least partially flexible. PCB wrap 204 may be positioned between the lubricious layer and the outer jacket braid(s) of the shaft 202. The PCB wrap 204 may be positioned around the exterior of the shaft 202 in a spiral arrangement or configuration. The PCB wrap 204 may be positioned on top of (e.g., radially exterior of) a lubricious layer of the shaft 202, and one or more coverings or coating may be applied to the PCB wrap 204 to help secure the PCB wrap 204 to the shaft 202. Alternatively or additionally, the PCB wrap 204 may be positioned between the lubricious layer and one or more outer braids of the shaft 202. In some embodiments, the outer braids may include one or more electrical shield layers and/or a reference plane, for example, for impedance control.

The outer flex PCB wrap 204 includes proximal electrical connections 216A and distal electrical connections 216B. The proximal electrical connections 216A are coupled to the handle 12, and the distal electrical connections 216B are coupled to the distal tip 20. The distal tip 20 may include a distal cap (discussed below) that may connect to the distal end 212 of the shaft 202. The proximal electrical connections 216A and the distal electrical connections 216B may together be referred to as “electrical connections 216A, 216B” herein. Electrical connections 216A, 216B may include suitable electrical connectors, including pins, plugs, ports, solder connections, etc. The proximal electrical connections 216A and distal electrical connections 216B may connect to the handle 12 and the distal tip 20, respectively, using any suitable electrical connection(s).

The electrical connections 216A, 216B may connect to electrical communication conduits 232 and/or optical circuitry extending from the proximal end 214 of the shaft 202 to the distal end 212 of the shaft 202. The electrical communication conduits 232 and/or optical circuitry may include one or more of cables, wires, optical fibers, and/or illumination fibers for transmitting electrical signals, such as, for example, image sensor data, imager instruction signals, and power between the handle (e.g., handle 12 in FIGS. 1A and 1B) and the distal end 212 of the shaft 202. For example, the electrical connections 216A, 216B may provide image processing (e.g., amplification, filtering, etc.), power, and/or electricity to the distal tip 20 (FIG. 1A).

The PCB wrap 204 may be positioned around the shaft 202 in various pitches and/or other arrangements, for example, to help accommodate different flexibilities of different portions of the shaft 202. For example, a portion of PCB wrap 204 may wrap around a portion of the shaft 202 with a larger longitudinal spacing between portions of the PCB wrap 204 to allow respective portion(s) of the shaft 202 to have more flexibility. For example, the portion of PCB wrap 204 may be positioned with a larger longitudinal spacing between portions of the PCB wrap 204 over the distal shaft portion 226 and the middle shaft portion 228 relative to other portions of PCB wrap 204. Additionally or alternatively, the PCB wrap 204 may be positioned with a smaller longitudinal spacing between portions of the PCB wrap 204 around the shaft 202 to provide less flexibility for respective portion(s) of the shaft 202. For example, a portion of PCB wrap 204 may be positioned with a smaller longitudinal spacing between portions of the PCB wrap 204 over the proximal shaft portion 230. The varied pitches and/or arrangements of the PCB wrap 204 around different portions of the shaft 202 may help to allow the PCB wrap 204 to meet the variable flexibility requirements. For example, the PCB wrap 204 may help to provide a more flexible and/or maneuverable distal portion 226 of the shaft 202, while also helping to provide a more rigid and/or stationary proximal portion 230 of the shaft 202.

A portion of the distal portion 226 of the shaft 202 may include an indentation or groove 208. For example, groove 208 may extend longitudinally through a radially outer portion of the distal portion 226. In these aspects, groove 208 may extend radially inward relative the outer surface 234 of the distal portion 226. The groove 208 may be created by forming a groove or indentation (e.g., radially inward) on the outer surface 234 the shaft 202. Additionally, the groove 208 may help to accommodate one or more electrical components (e.g., an integrated circuit 206) of the shaft 202. The groove 208 may be located on the distal shaft portion 226 (as shown), for example, extending to the distal end of shaft 202. Alternatively, although not shown, the shaft 202 may include one or more grooves 208 on the middle shaft portion 228 and/or the proximal shaft portion 230 of the shaft 202.

The groove 208 may have a size that allows for one or more integrated circuits 206 to be positioned within the groove 208 such that the one or more integrated circuits 206 do not extend radially outward of the groove 208 (e.g., forming a flat surface with the shaft 202). Additionally, the size and/or shape of groove 208 may provide clearance 224 (e.g., on sides of integrated circuit 206) to accommodate some change in the shape of the groove 208 when the shaft 202 is moved (e.g., deflected or manipulated through tortuous paths or lumens). For example, the groove 208 may have a depth that allows for the integrated circuit 206 to be placed in the groove 208, where the depth of the groove 208 is equal to or greater than the height of the integrated circuit 206. The groove 208 may also have a width and a length that are greater than the width and the length of the integrated circuit 206. The width of the groove 208 may also provide the clearance 224 to allow for minor changes in shape due to movement of the shaft 202.

One or more integrated circuits 206 may then be placed in the groove 208. One or more interfacing and/or signal conditioning integrated circuits (e.g., integrated circuit 206) may be located in line with the shaft 202. The integrated circuit 206 may also have passive components (e.g., capacitors and/or resistors) for power conditioning, gain, and filtering. In some embodiments, although not shown, the shaft 202 may include a plurality of grooves 208 to accommodate multiple integrated circuits. The shaft 202 may include a plurality of grooves 208 spaced circumferentially (e.g., evenly spaced or unevenly spaced) around the outer surface 234 of the distal portion 226 of the shaft 202.

As mentioned, the PCB wrap 204 may be wrapped around the shaft 202 in a spiral configuration. Additionally, the PCB wrap 204 may at least partially cover or enclose the integrated circuit(s) 206. For example, the integrated circuit 206 may be placed in the groove 208, and the PCB wrap 204 may be positioned around the shaft's 202 lubricious layer. In some embodiments, the lubricious layer may extend into the groove 208. Alternatively, in some embodiments, the groove 208 may extend radially through the lubricious layer. In some embodiments, the integrated circuit 206 is mounted on or otherwise coupled to the bottom layer or surface of the PCB wrap 204. When the PCB wrap 204 is placed on the shaft 202, the PCB wrap 204 may be positioned so that the integrated circuit 206 is positioned within the groove 208. In some aspects, as mentioned above, the integrated circuit 206 may fit flat on the shaft 202, for example, with the integrated circuit 206 being fully positioned within the groove 208.

The integrated circuit 206 may be connected to the flex PCB wrap 204 before, during, or after the wrapping of the PCB wrap 204. The wiring for the integrated circuit 206 may be connected to the electrical communication conduits 232 and/or optical circuitry of the flex PCB wrap 204 in any suitable fashion (e.g., physically and/or electrically connected). For example, the PCB wrap 204 may be connected to the integrated circuit 206 via an operator applying pressure (e.g., radially inward pressure) on the PCB wrap 204 and/or the integrated circuit 206, so that the integrated circuit 206 “snaps” to the flex PCB wrap 204, or vice versa. In some embodiments, the PCB wrap 204 may be connected to the integrated circuit 206 by applying an anisotropic conductive film (ACF) to the integrated circuit 206. In any of these aspects, a coating may be applied to the flex PCB wrap 204 and the shaft 202 to help secure the placement of the flex PCB wrap 204. The coating may include any kind of lubricous liner or any other kind of coating.

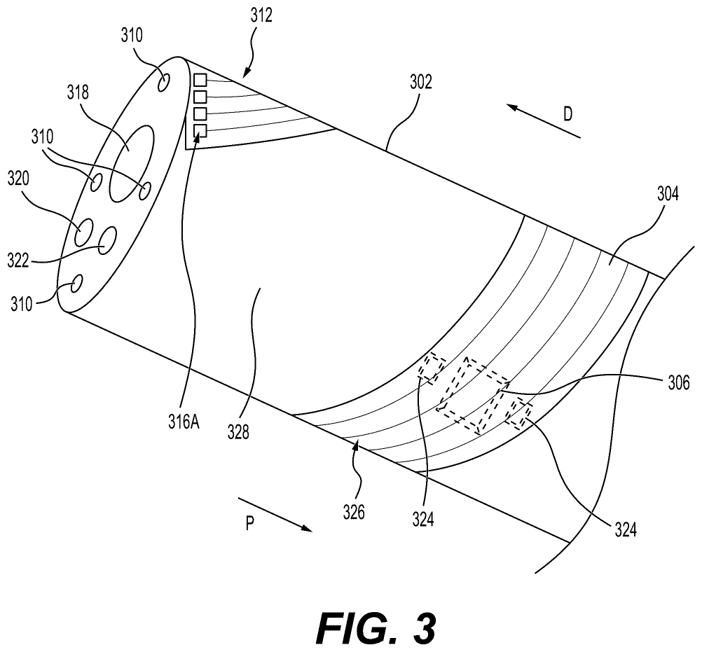

FIG. 3 depicts an embodiment of a shaft 302 that may be used as part of the shaft 2, in the embodiment shown in FIG. 1A. The shaft 302 may comprise the same (or similar) elements as the shaft 2 or the shaft 202 of FIGS. 1A and 2 respectively. For example, the shaft 302 may include one or more of steering wire lumens 310, a working channel 318, a suction lumen 320, and an irrigation lumen 322 to allow for, for example, air, water, suction, power, etc., to pass from the handle 12 (FIG. 1A), through the shaft 302, to the distal tip 20 (FIG. 1A). The shaft 302 may also include distal electrical connections 316A that connect to electrical communication conduits 326 and/or optical circuitry extending from the proximal end (not shown) of the shaft 302 to the distal end 312 of the shaft 302. Although FIG. 3 does not show proximal end, it is noted that the shaft 302 may include distal end electrical connections 316A and proximal end electrical connections (not shown), as discussed above. The shaft 302 also may include a PCB wrap 304, as described above.

The shaft 302 includes one or more integrated circuits 306 and/or one or more passive components 324 (e.g., capacitors and/or resistors). In some aspects, as shown, the shaft 302 may include one integrated circuit 306 and two passive components 324. One passive component 324 may be positioned proximal of the integrated circuit 306, and one passive component 324 may be positioned distal of the integrated circuit 306. The one or more integrated circuits 306 and/or the one or more passive components 324 may be positioned on an outer surface 328 of the shaft 302, for example, positioned on top of (e.g., radially outward of) the lubricious layer of the shaft 302. In some embodiments, the integrated circuit(s) 306 and the passive component(s) 324 may be coupled to the shaft 302 via a snap fit to the shaft 302. For example, the shaft 302 may include openings or indentations for the integrated circuit(s) 306 and the passive component(s) 324 to snap into, and an operator may apply pressure (e.g., radially inward pressure) to the integrated circuit(s) 306 and the passive component(s) 324 for them to snap-fit into the openings or indentations of the shaft 302.

The PCB wrap 304 may be wrapped over the integrated circuit 306 and/or the passive component(s) 324 to help secure the integrated circuit 306 and/or the passive components 324 to the shaft 302. The integrated circuit 306 and/or the passive component(s) 324 may also be connected (e.g., physically and/or electrically connected) to the PCB wrap 304 by any type of connection. For example, the integrated circuit 306 and/or the passive component(s) 324 may be soldered to the PCB wrap 304, and then the PCB wrap 304 may be positioned around the shaft 302. An operator may then apply pressure (e.g., radially inward pressure) to the integrated circuit 306 and/or the passive component(s) 324 for the integrated circuit 306 and/or the passive component(s) 324 to snap on or to otherwise connect to a cap containing a sensor or other circuits mating with distal electrical connections 316A.

In some embodiments, the integrated circuit 306 and the passive components 324 are mounted on a bottom layer or surface (e.g., closest to the body of the shaft 302) of the PCB wrap 304. When the outer flex PCB wrap 304 is secured to the shaft 302, the integrated circuit 306 and the passive components 324 may also be secured to the shaft 302. The integrated circuit 306 and the passive component(s) 324 may connect to the shaft 302 via any type of connection. For example, in some embodiments, the shaft 302 may include openings or indentations, and the integrated circuit(s) 306 and the passive component(s) 324 may snap into the openings or indentations. In these aspects, an operator may apply pressure to the integrated circuit 306 and the passive components 324, where, in response to the pressure, the integrated circuit 306 and the passive components 324 snap and attach to the shaft 302. Alternatively, the integrated circuit(s) 306 and the passive component(s) 324 may be secured to the shaft 302 by the PCP wrap 304 and/or the outer jacket braid(s).

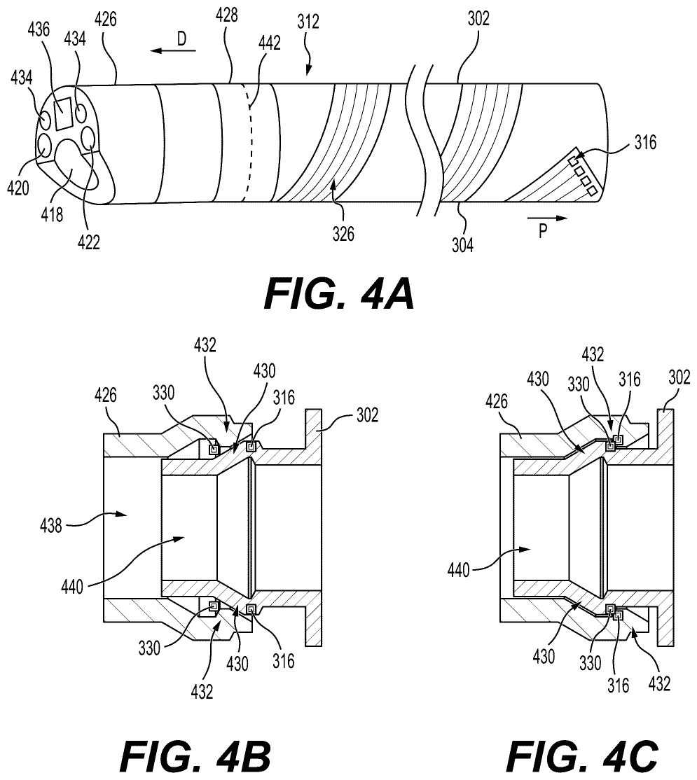

FIGS. 4A-4C illustrate various aspects of a distal cap module 426. For example, FIG. 4A depicts an embodiment of the distal cap module 426 that may be connected to the shaft 202 or the shaft 302. FIG. 4B illustrates the distal cap module 426 and the shaft 302 in a first configuration, and FIG. 4C illustrates the distal cap module 426 and the shaft 302 in a second configuration.

The distal cap module 426 may be attached (e.g., via a “snap fit”) and secured to the distal end of the shaft 302. For example, the distal cap module 426 may be removably attached to the shaft 302 (e.g., via the “snap fit”), and the distal cap module 426 may also secured to the shaft 302 by a shrink wrap 428. The shrink wrap 428 may span an interface 442 between the shaft 302 and the distal cap module 426. The shrink wrap 428 may be at least partially flexible. The shrink wrap 428 may be cut or otherwise removed to allow for the distal cap module 426 to be uncoupled from the shaft 302.

The distal cap module 426 may include various electrical connections (not pictured) to provide various functionality at the distal end. For example, the distal cap module 426 includes a cap working channel 418, a cap suction lumen 420, and a cap irrigation lumen 422. When the distal cap module 426 is coupled to the shaft 302, the working channel 322 is fluidly connected to the cap working channel 422. Additionally, the suction lumen 320 is fluidly connected to the cap suction lumen 420, and the irrigation lumen 322 is fluidly connected to the cap irrigation lumen 422. The distal cap module 426 may also include one or more lighting sources 434 (e.g., an LED) and an imaging device 436 (e.g., a camera). The lighting source(s) 434 and the imaging device 436 may receive the power to function via the handle 12 (e.g., from the umbilicus 16). In these aspects, the handle 12 may provide power distally through the proximal electrical connections 316B to the outer flex PCB wrap 304, via the distal electrical connections (not pictured) to the distal cap module 426. The electrical connections 316A (FIG. 3) and 316B may connect to electrical communication conduits 326 and/or optical circuitry extending from the proximal end of the shaft 302 to the distal end of the shaft 302. The electrical communication conduits 326 and/or optical circuitry may include one or more of cables, wires, optical fibers, and/or illumination fibers for transmitting electrical signals, such as, for example, image sensor data, imager instruction signals, and power between the handle 12 and the distal tip 312 of the shaft 302. For example, the electrical connections 316A and 316B may provide image processing (e.g., amplification, filtering, etc.), power, and/or electricity to the distal tip 312.

As mentioned above, FIGS. 4B and 4C are longitudinal cross-sectional views that depict aspects of the connection between the distal cap module 426 and the shaft 302. Specifically, FIG. 4B depicts the shaft 302 and the distal cap module 426 in the first or unconnected configuration, and FIG. 4C depicts the shaft 302 and the distal cap module 426 in a second or connected configuration.

As shown in FIG. 4B, the distal cap module 426 includes an opening 438 configured to receive a distal portion 440 of the shaft 302 via a securement region 432. The securement region 432 includes a radially inward extending projection that securely connects to the distal portion of the shaft 302. The shaft 302 is configured to fit into the opening 438 of the distal cap module 426. The shaft 302 may include a distal end portion 440 and flange 430, for example, spaced proximally of the distal end portion 440 of the shaft 302. The distal end portion 440 may include a smaller diameter or cross-section than a more proximal portion of the shaft 302. Additionally, the flange 430 may extend radially outward of the distal end portion 440 configured to interface with the securement region 432.

When the distal cap module 426 is coupled to the shaft 302, as shown in FIG. 4C, the shaft electrical connections 316B and the distal cap module electrical connections 330 meet and connect. As such, the handle 12 (FIG. 1A) may send power signals and/or control signals through the PCB wrap 304 and through the distal cap module 426. The power signals and/or control signals may control the lighting sources 434, the imaging device 436, and/or any other components.

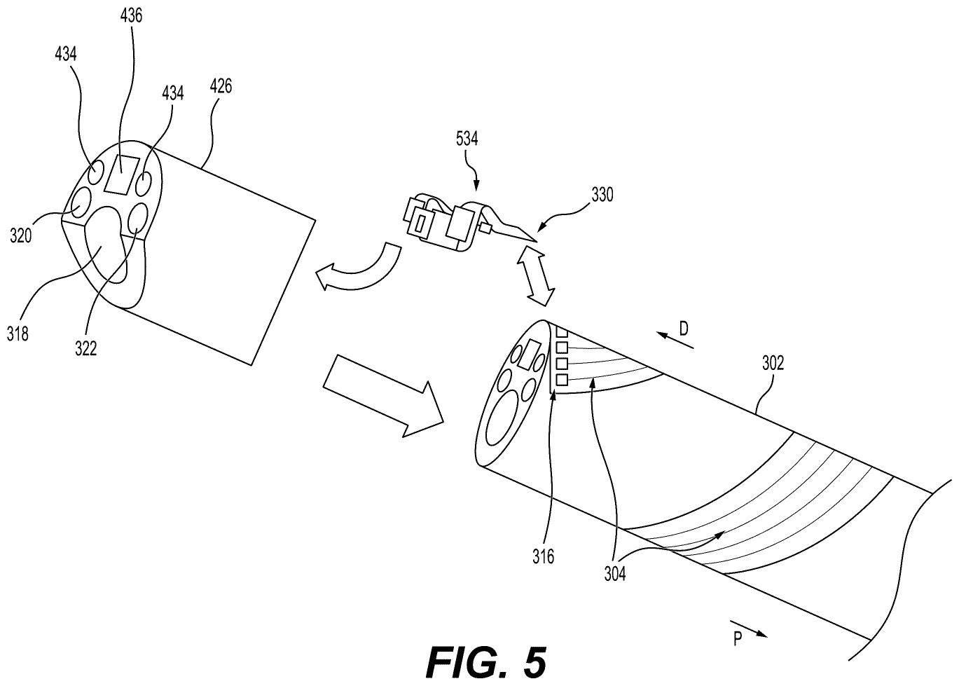

For example, as shown in FIG. 5, the distal cap module 426 includes a flexible printed circuit board (PCB) 534. When the distal cap module 426 is attached to the shaft 302, the electrical connections 330 of the flexible PCB 534 may connect to the electrical connections 316A of the PCB wrap 304. As a result, the power signals and/or control signals sent through the outer flex PCB wrap 304 are further provided through the distal cap module 426, and output and/or implemented via the lighting sources 434, the imaging device 436, and any other components. Although not shown, the proximal end of the shaft 302 may be coupled to the handle in a similar manner, such that the connections on the proximal end of the shaft 302 are connected to respective connections on the handle.

Embodiments of a shaft of a medical device including a groove for an integrated circuit and one or more passive components covered by a flex PCB wrap, as well as distal cap modules, can provide one or more of the following benefits. In some aspects, the ability to swap out a distal cap, shaft, and handle assemblies may help to reduce scrap costs during manufacturing. For example, manufacturing scopes may be challenging because cabling is brought through the long lumen of the shaft. As a result, catching issues downstream may result in costly scraps of entire assemblies. The ability to swap out the distal cap, shaft, and/or handle may help to identify issues earlier in the manufacturing process. Additionally, the ability to swap out a distal cap, shaft, and handle assemblies may help to simplify the final assembly process, for example, by minimizing the cable pulling requirements through the lumen of the shaft. Additionally or alternatively, as discussed above, varying the spacing or pitch of the PCB wrap around the shaft may help to provide extra stability and/or flexibility where needed on the shaft. Moreover, in some aspects, positioning the PCB wrap (e.g., including a plurality of communication conduits) outside of the lumen (i.e., rather than within the lumen) may help to allow for a larger working channel(s), in addition to other lumens, of the shaft.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. A medical device, comprising:
a shaft, wherein the shaft includes one or more lumens extending longitudinally from a proximal end of the shaft to a distal end of the shaft;
a printed circuit board, wherein the printed circuit board is flexible and is positioned around an outer surface of the shaft, wherein the printed circuit board includes a plurality of electrical connections at a proximal end of the printed circuit board, wherein the printed circuit board includes another plurality of electrical connections at a distal end of the printed circuit board, wherein the printed circuit board includes a plurality of communication conduits connecting electrical connections at the proximal end of the printed circuit board with respective electrical connections at the distal end of the printed circuit board; and
an end cap, wherein the end cap includes one or more cap lumens and a plurality of end cap electrical connections,
wherein, when the end cap is coupled to the shaft, (1) the one or more cap lumens are in communication with the one or more lumens in the shaft, and (2) the plurality of end cap electrical connections are electrically connected to the electrical connections at the distal end of the printed circuit board.

2. The medical device of claim 1, further comprising one or more integrated circuits coupled to and in electrical communication with the printed circuit board.

3. The medical device of claim 2, wherein a portion of the shaft includes a groove, and wherein the one or more integrated circuits are positioned within the groove.

4. The medical device of claim 3, wherein the groove has a width and a length that are greater than a width and a length of the one or more integrated circuits.

5. The medical device of claim 3, wherein the groove is positioned within a distal portion of the shaft.

6. The medical device of claim 3, wherein the printed circuit board is configured to connect to the shaft via an operator applying pressure on the printed circuit board, so that the one or more integrated circuits attached to the printed circuit board and the shaft snap together.

7. The medical device of claim 2, further comprising one or more capacitors or resistors coupled to and in electrical communication with the printed circuit board.

8. The medical device of claim 2, further comprising an anisotropic conductive film, wherein the anisotropic conductive film secures the one or more integrated circuits to the printed circuit board.

9. The medical device of claim 1, wherein the one or more lumens in the shaft includes a working channel, an irrigation lumen, a suction lumen, and a plurality of pull wire lumens.

10. The medical device of claim 9, further comprising:
a handle coupled to the proximal end of the shaft, wherein the handle includes a port fluidly connected to the working channel of the shaft.

11. The medical device of claim 1, wherein the end cap includes one or more lighting sources and an imaging device, wherein the one or more lighting sources and the imaging device receive power via the plurality of electrical connections at the distal end of the shaft.

12. The medical device of claim of claim 1, wherein the end cap includes an end cap printed circuit board that includes the plurality of end cap electrical connections.

13. The medical device of claim 1, wherein the plurality of communication conduits include one or more of: one or more cables, one or more wires, one or more optical fibers, or one or more illumination fibers for transmitting electrical signals.

14. The medical device of claim 1, wherein the end cap is configured to removably attach to the distal end of the shaft via a snap fit.

15. The medical device of claim 1, wherein a portion of the printed circuit board is positioned around the outer surface with a larger longitudinal spacing between at least two portions of the printed circuit board, and wherein a different section of the printed circuit board is positioned around the outer surface with a smaller longitudinal spacing between at least two other portions of the printed circuit board.

16. A medical device, comprising:
a shaft, wherein the shaft includes one or more lumens extending longitudinally from a proximal end of the shaft to a distal end of the shaft, wherein a portion of the shaft includes a groove, and wherein one or more integrated circuits are positioned within the groove; and
a flexible printed circuit board positioned around an outer surface of the shaft, the flexible printed circuit board including a plurality of proximal electrical connections on the proximal end of the shaft and a plurality of distal electrical connections on the distal end of the shaft, wherein a plurality of electrical communication conduits connect the plurality of proximal electrical connections and the plurality of distal electrical connections.

17. The medical device of claim 16, wherein the shaft includes a lubricious layer around the outer surface of the shaft.

18. The medical device of claim 16, wherein the groove extends radially inward relative to the outer surface of the shaft, and wherein the groove has a width and a length that are greater than an integrated circuit width and an integrated circuit length of the one or more integrated circuits.

19. A medical device, comprising:
a shaft, wherein the shaft includes one or more lumens extending longitudinally from a proximal end of the shaft to a distal end of the shaft, and wherein the shaft includes one or more electrical connections attached to an outer surface of the shaft; and
an end cap removably coupled to the distal end of the shaft, wherein the end cap includes one or more cap lumens and a plurality of end cap electrical connections,
wherein, when the end cap is coupled to the shaft, (1) the one or more cap lumens are in communication with the one or more lumens in the shaft, and (2) the plurality of end cap electrical connections are electrically connected to the one or more electrical connections of the shaft.

20. The medical device of claim 19, wherein the end cap includes a flexible printed circuit board, wherein, when the end cap is coupled to the shaft, one or more electrical connections of the flexible printed circuit board are electrically connected to the one or more electrical connections attached to the outer surface of the shaft.
